(19) United States
(12) Patent Application Publication
(10) Pub. No.: US 2026/0157501 A1
(43) Pub. Date: Jun. 11, 2026

(54) CAP FOR VESSELS OF COSMETICS

(71) Applicant: Kugil Co., LTD., Pyeongtaek-si (KR)

(72) Inventor: Jong-Suh CHOI, Hwaseong-si (KR)

(21) Appl. No.: 19/234,306

(22) Filed: Jun. 11, 2025

(30) Foreign Application Priority Data
Dec. 10, 2024 (KR) 10-2024-0183002

(51) Int. Cl. A45D 34/04; A61K 8/02

(52) U.S. Cl. CPC A45D 34/04; A61K 8/02; A61K 2800/87

(57) ABSTRACT

Provided is a cap for vessels of cosmetics comprising: an inner cap member having a cap thread portion on its inner surface, configured to be fastened to a container thread portion at the upper end of a container that holds a liquid or solution-type substance; a dropper tube coupled to the inner cap member for drawing in and dispensing the contents of the container; and an outer cap member rotatably coupled to the outer circumference of the inner cap member.

### BACKGROUND OF THE INVENTION

### Field of the Invention

The present invention relates to a cap for vessels of cosmetics, and more particularly, to an auto-pumping cap that enables the internal contents of the container to be pumped by rotating an outer cap member. The invention incorporates an elastic member made of synthetic resin into the cap structure, utilizing the elastic force of this member to draw liquid substances filled inside the container during pumping. The structure is designed such that, even if the button member is concealed or retracted within the outer cap during distribution or when not in use, the elastic member is not compressed, thereby preserving its elasticity. Furthermore, the structure allows the button member to emerge when the outer cap is rotated or be pressed directly by the user, so that the elastic force of the elastic member is utilized only during pumping. This ensures that the elastic force of the synthetic resin-based elastic member does not deteriorate even during long-term distribution and storage.

### Description of the Related Art

In general, women apply makeup to make their faces appear more radiant and beautiful.

Cosmetics can be categorized according to their functions into basic skincare products, makeup cosmetics, hair care products, fragrances, and functional cosmetics. These cosmetics are also classified by their form—such as creams, powders, or liquids—and are stored in containers appropriate to each form.

Cosmetic containers are designed to hold skincare, color cosmetics, or functional cosmetics so that they can be used by consumers. Typically, basic skincare products are stored in containers with either narrow or wide openings. In contrast, functional cosmetics are often used in very small amounts and tend to be relatively high in price. As such, not only the method of use but also the structure of the cap differs significantly from that of general cosmetic containers to ensure functionality.

Functional cosmetics are usually in liquid form and often undergo long-term distribution after being filled into containers. When a dropper integrated into the cap is used, the user typically presses a button on the cap to perform a pumping action that draws the liquid cosmetic into the dropper before use.

Typically, when the button member is pressed, the elastic member is compressed, and when the button is released, the elastic restoring force of the elastic member causes the button to move up and down, thereby allowing the liquid substance or solution inside the container to be drawn into the dropper for use.

At this time, the elastic member, which is elastically installed beneath the button member, remains in a constantly compressed state due to the consistent pressure applied by the button member, even when it moves up and down. As a result, during long-term distribution, the elastic force of the elastic member deteriorates, reducing the pumping capability and weakening the suction power of the liquid substance or solution inside the cosmetic container. Furthermore, if the user continues to use the product in this state over a long period, the continuous pressure applied by the button member increases the fatigue of the elastic member, gradually leading to a decline in its elasticity.

In particular, while most components of the cap for vessels of cosmetics are made of synthetic resin and are thus recyclable, the elastic member is often made of metal, which poses a challenge for proper separation and disposal, making recycling difficult.

Therefore, molding the elastic member from synthetic resin would offer environmental advantages due to ease of recycling. However, as mentioned above, continuous compression caused by the button component results in faster deterioration of the elastic force in the synthetic resin elastic member, making it impractical for long-term use.

### SUMMARY OF THE INVENTION

The present invention has been devised to solve the aforementioned problems. Its objective is to provide a cap for vessels of cosmetics that prevents the elastic member from being compressed by the button member during distribution or storage—even when the elastic member is made of metal or synthetic resin—thereby maintaining zero fatigue on the elastic member and preventing the deterioration of its elasticity over time.

The above objective is achieved by a cap (200) for vessels of cosmetics that includes:
an inner cap member (210) having a cap screw part (212) formed on its inner surface, and fastened to the container screw part (102) at the upper part of a container (100) configured to hold a liquid or solution-based substance;
a dropper tube (230) connected to the inner cap member (210), which draws the substance from inside the container (100) and dispenses it; and
an outer cap member (220) rotatably coupled to the outer circumference of the inner cap member (210).

In this cap (200) for vessels of cosmetics, the following components are included:
a pump tube (214), which is formed to pass through the upper part of the inner cap member (210) toward the opening (100a) of the container (100) and is shaped as a tubular structure protruding upward to a certain length;
an elastic member (240), inserted into the inner cap member (210) and seated on a support portion (216) formed on the outer circumference of the inner cap member (210), with helical-shaped guide grooves (242) of a certain length symmetrically formed on both sides at the upper part;
a button member (250), having guides (252) protruding to fit into the guide grooves (242), and configured to move up and down through a through-hole (222) formed at the upper part of the outer cap member (220), guided in the helical direction of the grooves depending on the rotation direction;
a rotation mechanism for the button member, which rotates the button member (250) by rotating the outer cap member (220) coupled to the inner cap member (210);
   A fixed sealing part (260), fitted and joined to the upper end of the dropper tube (230) inserted into the pump tube (214), having a pumping hole (262) that communicates with the opening (232) at the upper part of the dropper tube (230), and tightly contacting the inner diameter of the pump tube (214); and
a movable sealing part (270), fitted to a pressing part (254) protruding from the bottom of the button member (250), and configured to move up and down in close contact with the inner diameter of the pump tube (214) in accordance with the vertical movement of the button member (250).

The invention is preferably configured such that the means of rotation for the button member comprises:
upper open type rotation grooves 218, formed on both sides of the outer surface of the inner cap member 210, and actuators 224, inserted into the rotation grooves 218 and interfere with the guides 252 of the button 250.

In addition, the rotation mechanism of the above button member preferably includes:
open-top rotation grooves (218) formed with a certain width on both sides of the outer circumferential surface of the inner cap member (210); and
operation parts (224) protruding from the inner circumferential surface of the outer cap member (220), inserted into the rotation grooves (218), and positioned to interfere with the guides (252) of the button member (250).

In addition, the cap preferably includes a separation-prevention step (226) that is formed to extend downward from the through-hole (222) at the upper part of the outer cap member (220), preventing the elastic member (240) or button member (250) from becoming detached.

Furthermore, it is preferable that the elastic member (240) is formed of either synthetic resin or metal.

### Advantageous Effect

The present invention provides the effect of maintaining the fatigue on the elastic member at zero by preventing it from being compressed during distribution or storage, even when the elastic member is molded from metal or synthetic resin. This solves the problem of reduced elasticity in the elastic member, thereby enabling functional use without performance loss when in use. Furthermore, if the elastic member is molded from synthetic resin, all components of the cap for vessels of cosmetics can be made from synthetic resin, allowing the entire cap to be recycled without separating the elastic member, thus offering an eco-friendly advantage.

### BRIEF DESCRIPTION OF THE DRAWINGS

FIG. 1 is a cross-sectional view showing the overall structure of the cap for vessels of cosmetics according to the present invention.

FIG. 2A is a perspective view (with the outer cap member omitted) showing a state where the outer cap member of the cap for vessels of cosmetics according to the present invention has not been rotated, and the guide of the button member is inserted and seated beneath the guide groove of the elastic member.

FIG. 2B is a perspective view (with the outer cap member omitted) showing a state where the outer cap member has been rotated, and the guide of the button member has rotated and moved to be seated at the upper part of the guide groove of the elastic member.

FIG. 3A is a front view corresponding to FIG. 2A (outer cap member omitted).

FIG. 3B is a front view corresponding to FIG. 2A (outer cap member omitted).

FIG. 4A is a cross-sectional view showing the structure in the state of FIG. 2A.

FIG. 4B is a cross-sectional view showing the structure in which the button member is pressed downward from the state of FIG. 4A to perform pumping.

### DETAILED DESCRIPTION OF THE INVENTION

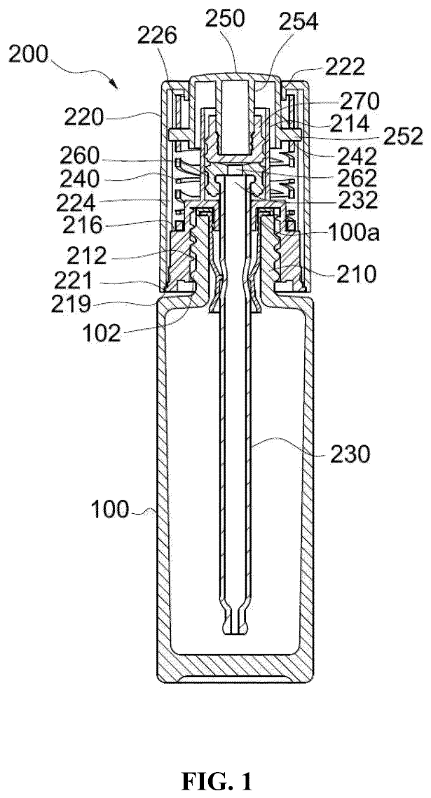

The structure of the cap for vessels of cosmetics according to the present invention will be described below with reference to the attached FIGS. 1 through 4B:

As shown in FIG. 1, the present invention comprises an inner cap member (210) that can be detachably fastened to the opening (100a) of the container (100), an outer cap member (220) coupled to the inner cap member (210), an elastic member (240) seated in the inner cap member (210), and a button member (250) that is vertically movable either by pressing down on the elastic member (240) or by the restoring force of the elastic member (240).

First, the cap thread portion (212) of the inner cap member (210) is fastened to the threaded portion (102) at the upper part of the container (100), and a pumping tube (214) is formed at the upper portion of the inner cap member (210).

The pumping tube (214) is a tubular structure that extends through an upper part of the inner cap member (210) toward the opening (100a) of the container (100) and is formed to extend upward by a predetermined length.

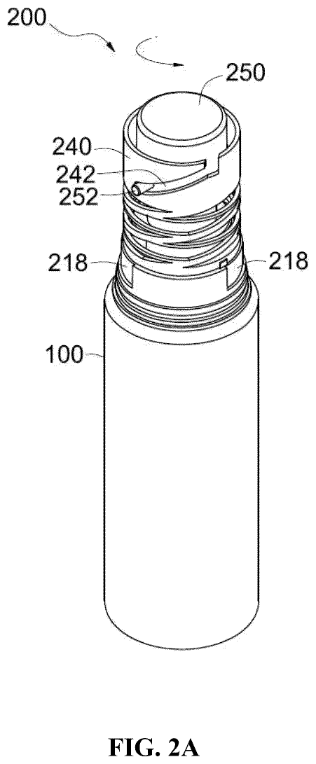

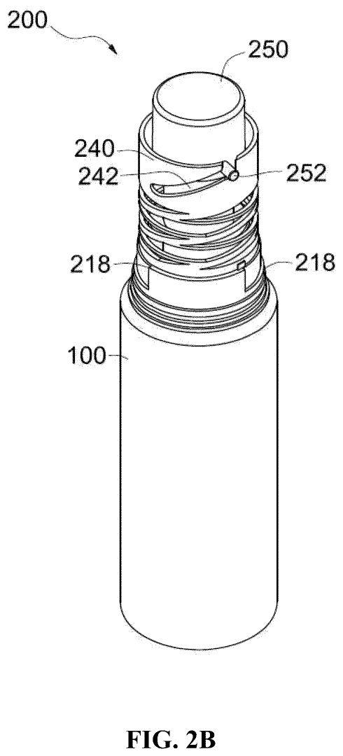

In addition, as shown in FIGS. 2A through 4B, a stepped support portion (216) is formed on the outer circumference of the inner cap member (210), and the elastic member (240), which is inserted around the outer circumference of the pumping tube (214), is seated on the support portion (216) in an upright position.

Here, the elastic member (240), as shown in the drawings, has helical guide grooves (242) formed on both sides of its upper portion. These guide grooves (242) are intended to determine the rotation radius and vertical travel height of the button member (250), which will be described later. The length, rotation radius, and rotation angle of the guide grooves can be varied and are not limited to specific values.

That is, when the button member (250) rotates, the guides (252) protruding from both sides of the button member (250) follow the guide grooves (242) as they move vertically. The button member (250) can rotate by more or less than 90°, depending on its height, which in turn affects the pumping action through the dropper tube (230). Therefore, these design parameters can be flexibly selected and changed based on the intent of the designer or manufacturer.

The outer circumference of the inner cap member (210) is configured such that the outer cap member (220) is fitted and coupled. Specifically, a locking groove (221) formed on the inner circumferential surface at the lower end of the outer cap member (220) is engaged with a locking protrusion (219) formed on the outer circumference at the lower end of the inner cap member (210), thereby coupling the outer cap member (220) to the inner cap member (210).

In addition, as shown in FIG. 1, a dropper tube (230) is inserted and coupled inside the lower portion of the pumping tube (214). A fixed sealing unit (260), which possesses elasticity to closely adhere to the inner wall of the pumping tube (214), is inserted and coupled to the upper end of the dropper tube (230). The fixed sealing unit (260) is provided with a pumping hole (262) formed to open upward and communicate with the upper opening (232) of the dropper tube (230).

Furthermore, a movable sealing unit (270) is inserted and coupled into a pressing part (254) protruding from the bottom of the button member (250). As the button member (250) moves up and down, the movable sealing unit (270) moves correspondingly while remaining in close contact with the inner surface of the pumping tube (214). This allows the contents inside the container (100) to be drawn in or discharged through the opening (232) of the dropper tube (230) and the pumping hole (262).

To prevent the components inside the outer cap member (220), such as the elastic member (240) and the button member (250), from becoming detached, it is preferable that an anti-separation ledge (226) is formed extending downward from the upper through-hole (222) of the outer cap member (220).

As described above, when the inner cap member (210), outer cap member (220), elastic member (240), and button member (250) are assembled, the guide (252) of the button member (250) is seated at the lower end of the guide groove (242), as shown in FIG. 1. At this point, the button member (250) is in a lowered state within the through-hole (222) of the outer cap member (220); however, since the button member (250) is not pressing down on the elastic member (240), the elastic member (240) remains in a relaxed, uncompressed state.

Ultimately, as the elastic member (240) is maintained in an uncompressed and relaxed state during distribution, even if the distribution period is extended, no fatigue is applied to the elastic member (240). Therefore, the elastic force of the elastic member (240) can be preserved in the same state as when it was manufactured.

Moreover, when the product is used by the consumer after the distribution period, the button member (250) is first raised through the through-hole (222) and then pressed downward. This enables pumping action using the elastic force and restoring force of the elastic member (240), which has not been degraded, ensuring a satisfactory and functional user experience.

Meanwhile, the present invention provides a means of rotation for the button member (250), which allows the button member to rotate when the outer cap member (220), coupled to the inner cap member (210), is rotated.

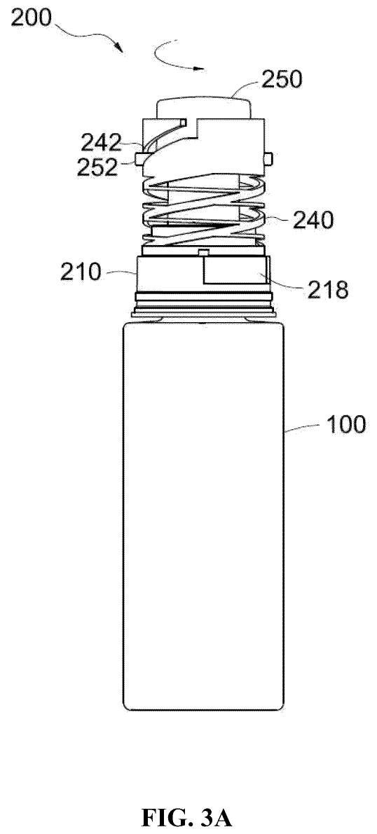

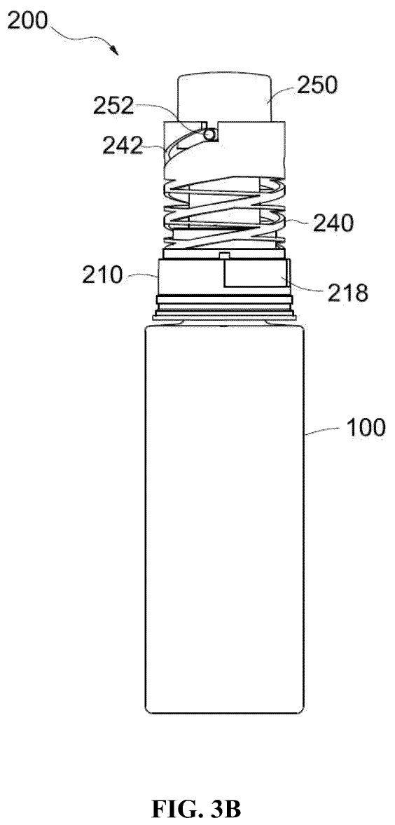

This means of rotation for the button member includes a rotation groove (218) with an open-top structure formed on both outer side surfaces of the inner cap member (210), as shown in FIGS. 2A and 3A. A protruding actuator (224) is formed on the inner circumferential surface of the outer cap member (220) so as to be inserted into the rotation groove (218). As shown in FIG. 1, this actuator (224) is inserted into the guide groove (242) of the elastic member (240) and interferes with the guide (252) of the button member (250) that protrudes outward.

Thus, when the outer cap member (220) is rotated, as illustrated in FIGS. 2A and 3A, the actuator (224) rotates within the rotation groove (218). While the inner cap member (210) remains stationary, the actuator (224) interferes with the guide (252), thereby rotating the guide (252) and the button member (250) together.

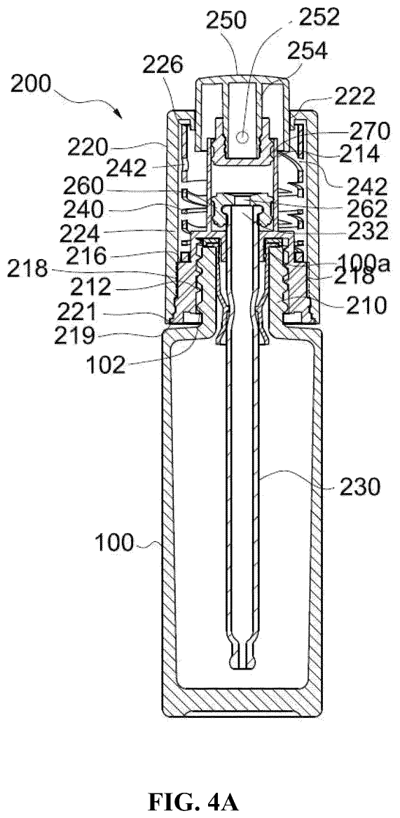

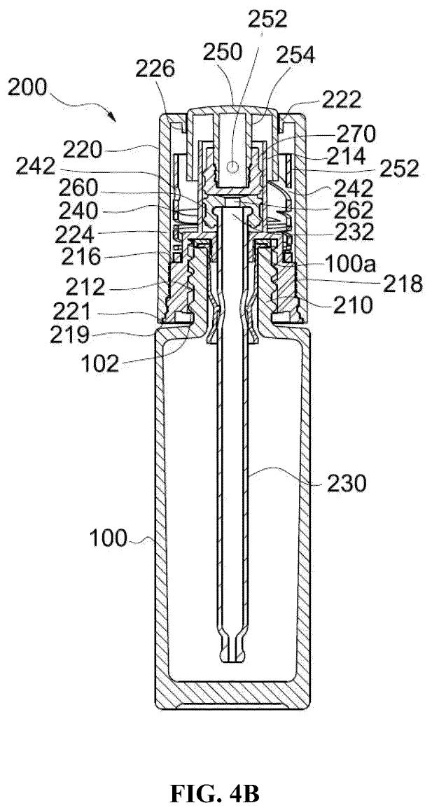

As a result, the guide (252) and the button member (250) move up or down along the helical guide groove (242) as they rotate, as shown in FIGS. 2B, 3B, and 4A. When the button member (250) is pressed downward, as shown in FIG. 4B, the outer cap member (220) and the inner cap member (210) remain fixed and do not rotate. Only the button member (250) descends, compressing the elastic member (240) downward. This causes the movable sealing unit (270), which is coupled to the pressing part (254), to descend to the lower part of the pumping tube (214), performing the pumping action.

At this point, if the actuator (224) on the outer cap member (220) moves beyond the rotation radius of the rotation groove (218), the actuator (224) interferes with the rotation groove (218), causing the inner cap member (210) to rotate. Consequently, the cap thread (212) of the inner cap member (210) and the container thread (102) of the container may be engaged or disengaged.

Therefore, in order to move only the button member (250) up and down, the actuator (224) must be operated within the rotation radius of the rotation groove (218).

As described above, after the user has completed the use, such as pumping and discharging, if the outer cap member (220) is rotated in the reverse direction at the user's discretion, the guide (252) rotates along the guide groove (242), causing the button member (250) to descend. Even in this case, since the button member (250) does not press the elastic member (240) while descending, the elastic force of the elastic member (240) is not degraded.

Here, when the outer cap member (220) is rotated with the button member (252) in the lowered position, the guide (252) rotates along the guide groove (242), causing the button member (252) to move up or down. At this time, the movable sealing unit (270), coupled to the pressing part (254) of the button member (252), also moves up or down. As a result, the contents inside the container (100) are drawn into the dropper tube (230), and this enables an auto-pumping function even without using the elastic force of the elastic member (240).

Of course, if the user wishes to use more of the contents inside the container (100), the user may rotate the outer cap member (220) in the reverse direction to lower the button member (252), then rotate it forward again to raise the button member (252), thus using the auto-pumping function. Alternatively, a simple pumping action can be performed by directly pressing the button member (252) downward to compress the elastic member (240), and then allowing the button member (252) to rise using the restoring force of the elastic member (240), drawing the contents through the dropper tube (230). These operations can be selectively performed depending on the user's preference.

The present invention was devised to eliminate the factor that degrades the elastic force of the elastic member (240) by increasing its fatigue. Even if the elastic member (240) is made of metal or synthetic resin, the invention fundamentally prevents the reduction in elasticity, allowing for long-term use.

Furthermore, if the elastic member (240) is molded from metal, it must be separated for recycling after use, which is very cumbersome and inconvenient, often leading to its disposal. However, if the elastic member (240) is molded from synthetic resin, since all the components of the cap for vessels of cosmetics according to the present invention are made of synthetic resin, the cap can be recycled without having to separate the elastic member (240), thereby offering an eco-friendly advantage.

What is claimed is:

1. A cap for vessels of cosmetics, the cap comprising an inner cap member having a cap screw portion formed on an inner circumferential surface thereof and fastened to a container screw portion at an upper part of a container configured to hold a liquid or fluid content, a dropper tube coupled to the inner cap member and configured to suction the liquid or the fluid contents inside the container and discharge the suctioned liquid or fluid contents, and an outer cap member rotatably coupled to an outer circumference of the inner cap member,
the cap further comprising:
a pumping tube having a tubular structure, penetrating from an upper part of the inner cap member toward an opening of the container, and protruding upward to a predetermined length;
an elastic member inserted into the inner cap member and seated on a support portion formed on the outer circumference of the inner cap member, the elastic member having symmetrical helical guide holes with a predetermined length on both upper sides of the elastic member;
a button member including guides that protrude to be inserted into the helical guide holes, the button member configured to be guided in a helical direction of the helical guide holes and configured to be moving up and down through a through-hole formed at an upper part of the outer cap member;
a rotator for the button member, configured to rotate the button member by rotating the outer cap member coupled to the inner cap member;
a fixed sealing part inserted and coupled to an upper end of the dropper tube inserted into the pumping tube, having a pumping hole such that an opening at an upper part of the dropper tube communicates with the pumping hole, the fixed sealing part being tightly adhered to an inner surface of the pumping tube; and
a movable sealing part inserted and coupled to a pressing part protrudingly formed at a lower part of the button member, the movable sealing part configured to be moving up and down while maintaining contact with the inner surface of the pumping tube.

2. The cap of claim 1, wherein the rotator comprises:
rotation grooves with an open-top structure, having a predetermined width on both outer side surfaces of the inner cap member; and
an operating part protrudingly formed on an inner circumferential surface of the outer cap member, the operating part inserted into the rotation grooves and configured to interfere with the guides of the button member.

3. The cap of claim 1, wherein the elastic member is made of a synthetic resin material or a metal material.
